(19) United States
(12) Patent Application Publication
(10) Pub. No.: US 2006/0239985 A1
(43) Pub. Date: Oct. 26, 2006

(54) Method of preparing cell for transplantation

(75) Inventors: Jeffrey D. Croissant, Scituate, MA (US); Hee-Won Yoo, Scoul (KR)

Correspondence Address: CLARK & ELBING LLP, 101 FEDERAL STREET, BOSTON, MA, 02110, US

(73) Assignee: Cardio3 S.A., Braine Alleund (BE)

(21) Appl. No.: 10/542,757

(22) PCT Filed: Dec. 30, 2003
(86) PCT No.: PCT/KR03/02898

§ 371 Date: Apr. 11, 2006

(30) Foreign Application Priority Data
Jan. 23, 2003 (KR) 10-2003-0004565

(51) Int. Cl. A61K 48/00; C12N 5/08; C12N 15/09

(52) U.S. Cl. 424/93.21; 435/455; 435/372

(57) ABSTRACT

The invention features a method of producing cells for transplantation into myocardial tissue of a mammal and a method for treating a disorder using the cells. The method comprises the steps: (a) providing bone marrow stem cells that have not been immortalized; (b) culturing said bone marrow stem cells in a culture medium containing IGF-1 (Insulin-like Growth Factor-I) under conditions that induce said cells to differentiate into cardiomyogenic cells; (c) monitoring the differentiation state of the cells of step (b); and (d) collecting the cells of step (b) when at least about 50% of said cells are cardiomyogenic cells. According to the method, the highest yield of the cells to be transplanted into mammal cardial tissue which have the characteristics of cardiomyocyte cell lineage mostly can be obtained. The cells produced as such can be used for treating a disorder characterized by insufficient cardiac function.

### TECHNICAL FIELD

The present invention relates to a method of producing cells for transplantation and a method for treating a disorder using the cells produced therefrom.

### BACKGROUND ART

The possibility of bone marrow being an in vivo source of circulating cardiomyocyte progenitors has been suggested. In one experiment, transplanted bone marrow-derived cells were observed to be distributed in a dystrophic mouse heart. Although the molecular characteristics of these cells were not identified, their location in the heart tissue indicated these cells were cardiomyocytes.

The ability of bone marrow mesenchymal stem cells (BMSCs) to differentiate as beating cardiomyocytes following introduction of inductive agents such as 5-azacytidine has also been shown. Based on these findings, BMSCs have been proposed to be a source of cells for treatment of cardiac disease and cardiac abnormalities.

Despite the potential therapeutic value of BMSCs, current cell transplantation methods for cardiac tissue are clinically inadequate because rate of implant incorporation into the host tissue is poor. For example, Orlic et al. (Nature 410: 701-705, 2001) reported that only 40% of mice receiving BMSC transplants showed some myocardial repair.

PCT Publication No. WO 02/083864, owned by ANTEROGEN CO., LTD., describes methods and reagents for cell transplantation, in which a method for producing cells for transplantation into myocardial tissue of a mammal comprises the following steps: (a) providing bone marrow stem cells that have not been immortalized; (b) culturing said bone marrow stem cells under conditions that induce said cells to differentiate into cardiomyogenic cells; (c) monitoring the differentiation state of the cells of step (b); and (d) collecting the cells of step (b) when at least about 10% and as many as 100% of said cells are cardiomyogenic cells.

In the cell transplantation, it is most important that cardiomyogenic cells are produced from bone marrow stem cells at high yield. However, the above method described in WO 02/083864 has the problem of low productivity.

Thus, there is a need for cell transplantation methods having high rates of cell incorporation and cell survival.

### DISCLOSURE OF THE INVENTION

It is an object of the present invention to provide a method of producing cells for transplantation into myocardial-tissue of a mammal at high yield, and a method for treating a disorder characterized by insufficient cardiac function using the cells produced therefrom.

In accordance with one aspect of the present invention, it is provided a method for producing cells for transplantation into myocardial tissue of a mammal comprising the steps:

(a) providing bone marrow stem cells that have not been imnortalized;

(b) culturing said bone marrow stem cells in a culture medium containing IGF-1 under conditions that induce said cells to differentiate into cardiomyogenic cells;

(c) monitoring the differentiation state of the cells of step (b); and

(d) collecting the cells of step (b) when at least about 50% of said cells are cardiomyogenic cells.

In a second aspect, the invention features a method for treating a disorder characterized by insufficient cardiac function in a mammal, comprising the steps:

(a) isolating bone marrow stem cells from said mammal;

(b) culturing said bone marrow stem cells in a culture medium containing IGF-1 under conditions that induce said cells to differentiate into cardiomyogenic cells;

(c) monitoring the differentiation state of the cells of step (b);

(d) collecting the cells of step (b) when at least about 50% of said cells are cardiomyogenic cells; and

(e) transplanting said cardiomyogenic cells into said mamrnal.

The present inventors have discovered that the rate of differentiation of bone marrow stem cells into cardiomyogenic cells may be maximized when the bone marrow stem cells are cultured in a medium containing IGF-1 (Insulin-like Growth Factor-1) in producing cells for transplantation into myocardial tissue of a mammal. The cells cultured in the medium containing IGF-1 show MEF2 expression more strongly, which means that the cells have the intense characteristics of cardiomyogenic cells.

In the method of the present invention, the cells can be human, pig, or baboon BMSCs. The transplantation can be an autologous transplantation, i.e., cells from the mammal to be treated are preferably transplanted. Preferably, at least about 15%, 20%, 30%, 40%, or 50% of the cells collected are cardiomyogenic cells (e.g., cardiomyocyte progenitor cells). Preferably, as many as about 60%, 70%, 80%, 95%, or 99% of the cells collected are cardiomyogenic cells. Most preferably, the cells are collected when at least about 50% and as many as about 80% of the collected cells are cardiomyogenic cells.

In another aspect, the invention features a method for treating a mammal (e.g., a human) diagnosed as having a disorder characterized by insufficient cardiac function. This method comprises the steps of introducing to the myocardial tissue of the mammal the following three types of cells: (1) cardiomyocytes or cardiomyocyte progenitor cells; (2) endothelial cells or endothelial cell progenitors; and (3) vascular smooth muscle cells or vascular smooth muscle cell progenitors in amounts sufficient to improve cardiac function.

In the present invention, for each injection, one million (1×10⁶) cardiomyocyte progenitor cells are injected into myocardium with the other two cell types in a ratio of about 10:1:1 (cardiomyocyte progenitors:endothelial cell progenitors:vascular smooth muscle cell progenitors). Other ratios can also be used.

There are numerous methods known in the art to induce cells to become cardiomyogenic. For example, the BMSCs can be cultured in culture medium that includes a cardiomyogenic cell-inducing amount of BMP-2 (Bone Morphogenic Protein 2) or bFGF (basic Fibroblast Growth Factor). The present invention is characterized in that the rate of differentiation of BMSCs into cardiomyogenic cells is maximized by adding variable concentrations of IGF-1 to the BMSCs for inducing the cells to differentiate into cardiomyogenic cells. In the present invention, IGF-1 can be added to the medium in the concentration ranging from 100 pg/ml to 25 ng/ml. These methods may be employed in the practice of the invention.

Because mitotic cells will likely integrate into the myocardium more easily than will postmitotic cells, it is desirable that at least about 25%, 50%, 75%, 90%, 95% or more of the transplanted cells of step (c) be mitotic progenitor cells.

In the method of the present invention, for improved generation of blood vessels, it is desirable that cardiomyocytes or cardiomyocyte progenitors be transplanted in amounts sufficient to improve cardiac finction. Preferably, the cells are derived from stem cells (e.g., BMSCs). Additionally, to facilitate the survival of the transplanted cells of invention, anti-apoptotic agents such as caspase inhibitors (e.g., zVAD-fmk) can be adrnistered with the injected cells.

The present invention also features a method for producing cells for transplantation into a mammal (e.g., a human). The method includes the steps of (a) providing a population of BMSCs; (b) culturing the cells under conditions that induce the cells to adopt a cell type selected fiom the group consisting of a vascular smooth muscle cell, an endothelial cell, an epicardial cell, an adipocyte, an osteoclast, an osteoblast, a macrophage, a neuronal progenitor, a neuron, an astrocyte, a skeletal muscle cell, a smooth muscle cell, a pancreatic precursor cell, a pancreatic β-cell, and a hepatocyte; (c) monitoring the state of differentiation of the cells of step (b); and (d) collecting the cells of step (b) when al least about 50% of the cells are expressing detectable amounts of a protein that is specific for the induced cell type. Appropriate markers are described herein. The bone marrow stem cells can be human, pig, or baboon BMSCs.

In one embodiment, the method includes the step of (e) transplanting the cells of step (d) into a mammal (e.g., a human). The transplantation can be an autologous transplantation, i.e., the cells are transplanted into the mammal from which the bone marrow stem cells were derived. The culturing and monitoring steps (b) and (c) are performed until at least about 15%, 20%, 30%, 40%, or 50% and as many as about 60%, 70%, 80%, 90%, 95%, or 99% of the cells express detectable amounts of the marker of the desired lineage. Preferably, the culturing and monitoring (b) and (c) are performed until at least about 50% and as many as about 80% of the cells express detectable amounts of the marker of the desired lineage.

The present invention also features a method for treating a disorder characterized by insufficient cardiac finction in a mammal, preferably a human. The method includes the steps of (a) isolating bone marrow stem cells from the mammal to be treated; (b) culturing the bone marrow stem cells under conditions that induce the cells to differentiate into cardiomyogenic cells; (c) monitoring the state of differentiation of the cells of step (b); (d) collecting the cells of step (b) when at least about 10% and as many as about 100% of the cells are cardiomyogenic cells; and (e) transplanting the cardiomyogenic cells into the mammal.

As used herein, by “stem cell” is meant a cell capable of (i) self renewing, and (ii) producing multiple differentiated cell types, including one of the group selected from cardiomyocyte, endothelial cell, and vascular smooth muscle cell.

By “bone marrow mesenchymal stem cell (BMSC)” is meant a bone marrow mesenchyme-derived stem cell that is CD45⁻. BMSCs are also referred to as “bone marrow stem cells” and “bone marrow multipotent progenitor cells”.

By “treating” is meant reducing or alleviating at least one adverse effect or symptom of a disorder characterized by insufficient cardiac function. Adverse effects or symptoms of cardiac disorders are numerous and well-characterized. Non-limiting examples of adverse effects or symptoms of cardiac disorders include: dyspnea, chest pain, palpitations, dizziness, syncope, edema, cyanosis, pallor, fatigue, and death. For additional examples of adverse effects or symptoms of a wide variety of cardiac disorders, see Robbins, S. L. et al. (1984) Pathological Basis of Disease (W. B. Saunders Company, Philadelphia) 547-609; and Schroeder, S. A. et al. eds. (1992) Current Medical Diagnosis & Treatment (Appleton & Lange, Connecticut) 257-356.

By “disorder characterized by insufficient cardiac function” includes an impairment or absence of a normal cardiac finction or presence of an abnormal cardiac function. Abnormal cardiac function can be the result of disease, injury, and/or aging. As used herein, abnormal cardiac function includes morphological and/or functional abnormality of a cardiomyocyte or a population of cardiomyocytes. Non-limiting examples of morphological and functional abnormalities include physical deterioration and/or death of cardiomyocytes, abnormal growth patterns of caridomyocytes, abnormalities in the physical connection between cardiomyocytes, under- or over-production of a substance or substances by cardiomyocytes, failure of cardiomyocytes to produce a substance or substances which they normally produce, transmission of electrical impulses in abnormal patterns or at abnormal times, and an altered chamber pressure resulting from one of the aforementioned abnormalities. Abnormal cardiac function is seen with many disorders including, for example, ischemic heart disease, e.g., angina pectoris, myocardial infarction, chronic ischemic heart disease, hypertensive heart disease, pulmonary heart disease (cor pulmonale), valvular heart disease, e.g., rheumatic fever, mitral valve prolapse, calcification of mitral annulus, carcinoid heart disease, infective endocarditis, congenital heart disease, myocardial disease, e.g., myocarditis, cardiomyopathy, cardiac disorders which result in congestive heart failure, and tumors of the heart e.g., primary sarcomas and secondary tumors.

“Administering,” “introducing,” and “transplanting” are used interchangeably and refer to the placement of the cardiomyogenic cells of the invention into a subject, e.g., a human subject, by a method or route which results in localization of the cells at a desired site.

By “cardiac cell” is meant a differentiated cardiac cell (e.g., a cardiomyocyte) or a cell committed to producing or differentiating as a cardiac cell (e.g., a cardiomyoblast or a cardiomyogenic cell).

By “cardiomyocyte” is meant a muscle cell in heart that expresses detectable amounts of cardiac markers (e.g., alpha-myosin heavy chain, cTnI, MLC2v, alpha-cardiac cardiac actin, and, in vivo, Cx43), contracts, and does not proliferate.

By “cardiomyoblast” is meant a cell that expresses detectable amounts of cardiac markers, contracts, and proliferates.

By “cardiomyogenic cell” is meant a cell expressing detectable amounts of MEF2 protein, and does not show organized sarcomeric structures or contractions, and preferably does not express detectable amounts of myosin heavy chain protein.

By “specifically induce one cell type” when referring to differentiation of cultured BMSCs is meant a culture wherein at least 50% of BMSCs differentiate into the desired cell type (i.e., cardiomyocytes).

By “detectable amounts” of a protein is meant an amount of a protein that is detectable by immunocytochemistry using, for example, the methods provided herein. One method for determining whether a cell is detectably labeled with either Csx/Nkx2.5 or myosin heavy chain is provided below. Cultured cells are fixed with 4% formaldehyde for 20 minutes on ice, then incubated for 15 minutes in 0.2% Triton X-100 in phosphate-buffered saline (PBS). After three washes in PBS, the cells are incubated in blotting solution (1% BSA and 0.2% Tween 20 in PBS) for 15 minutes. The samples are then treated with one of the following antibodies: anti-Csx (1:100-1:200, from S. Izuro, Harvard Medical School, Boston Mass.), MF-20 (1:50-1:200, from Developmental Studies Hybridoma Bank, University of Iowa, Iowa City Iowa), anti-desmin (1:100-1:200, from Sigma-Aldrich, Inc., St. Louis Mo.), and, if desired, their isotype controls (for Csx, normal rabbit serum; for MF-20, mouse IgG2b; for desmin, mouse IgG1) at the same concentration, and incubated overnight at 4° C. in a moist chamber. The sample slides are then washed three times using a washing solution (0.5% Tween 20 in PBS) and incubated with secondary antibodies (for Csx, donkey anti-rabbit IgG, for MF-20 and anti-desmin, donkey anti-mouse IgG, all from Jackson ImmunoResearch Laboratories, Inc.) following the instructions provided by the vendors, followed by three washes. The samples are then examined under a fluorescence microscope (e.g., a Nikon TS100 microscope with a matching fluorescence attachments) and visually scored for immunolabeling.

Other features and advantages of the present invention will be apparent from the following description of the preferred embodiments thereof, and from the claims.

The inventors discovered that transplanting developmentally committed but undifferentiated cells will improve the survival, incorporation, and adaptation of the implant in the target tissue.

The inventors also discovered a therapeutic cellular transplantation method in which blood vessels and myocardial tissue are collectively regenerated in the area of treated myocardium. This method includes the transplantation of undifferentiated cells committed to become one of three cell types: cardiomyocytes, endothelial cells, or vascular smooth muscle cells.

In the present invention, the BMSCs derived from a human are used to differentiate into cardiomyogenic cells.

It is desirable that there be an ample supply of the cells to be transplanted. Accordingly, in one aspect, the cells to be transplanted are derived from stem cells. One suitable stem cell is the BMSC, which can be isolated from adult bone marrow. Once isolated, BMSCs can be treated with growth factors (referred to herein as “priming”) to induce the cells toward a cardiomyocyte cell lineage, as is described below. In the present invention, for inducing the BMSCs to differentiate into a cardiomyocyte, variable concentrations of IGF-1 are added to the BMSCs and then the effect of IGF-1 is determined.

Optimization of stem cells and stem cell derivative preparations is critical for successful cell transplantation. To achieve maximum yield in cell transplantation, the implanted cells are desirably at the proper stage of commitment and differentiation.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a series of micrographs showing the staining and morphology of differentiated BMSCs derived from human using the antibodies specific for MEF2 (A), GATA (E) and desmin (I), following a co-culture with growth factors, compared with those of corresponding isotype (C, G, K) negative controls; and

FIG. 2 is a series of micrographs showing the staining and morphology of differentiated BMSCs derived from human using the antibody specific for MEF-2, following a co-culture with growth factors in the absence (A) or presence (C) of IGF-1.

### BEST MODE FOR CARRYING OUT THE INVENTION

Hereinafter, the present invention will be described in detail, in conjunction with various examples. These examples are provided only for illustrative purposes, and the present invention is not to be construed as being limited to these examples.

### EXAMPLE 1

### Method of Enhancing the Differentiation of BMSCs into Cardiomyogenic Cells

Marrow was isolated from adult human. The BMSCs were isolated and cultured in medium containing 10% fetal bovine serum, 100 μM L-ascorbic acid-2-PO₄, 5-15 ng/ml human LIF (leukemia inhibitory factor), and 20 nM dexamethasone. This in vitro condition allows the BMSCs to maintain their self-renewing character and to expand by passaging without losing responsiveness to the differentiation agents such as growth factors.

The BMSCs were cultured for 2 weeks in the presence of growth factors (50 ng/ml bFGF, from R&D and 25 ng/ml BMP-2, from R&D) and IGF-1 (2 ng/ml, from R&D). At which time, the cells were subjected to immunofluorescence staining using the markers specific for muscle cell, that is MEF2, GATA or desmin.

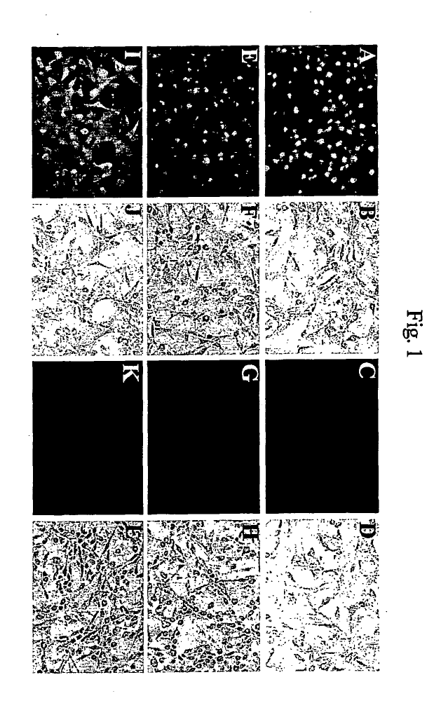

FIG. 1 is a series of micrographs showing the staining and morphology of differentiated BMSCs derived from human using the antibodies specific for MEF2 (A), GATA (E) and desmin (I), following a co-culture with growth factors, compared with those of corresponding isotype (C,G,K) negative controls. In FIG. 1, panels A, E and I are the results from the immunofluorescence staining by MEF2, GATA and desmin, respectively. Panels B, F and J show the corresponding phase contrast images of each immunofluorescent image. Panels C, G and K show the corresponding fluorescent images of the isotype negative controls. Panels D, H and L show the corresponding phase contrast images of each isotype control. All images were observed under 40× objective.

Subsequently, the BMSCs isolated from human were treated with either bFGF and BMP2, or bFGF, BMP2 and IGF-1. After exposure to differentiation media for one week, the cells were fixed and assayed for MEF2 immunofluorescence staining using a polyclonal antibody to MEF2 (Santa Cruz #sc-10794). The concentrations of bFGF, BMP2, and IGF-1 to be treated and the test conditions were same as above.

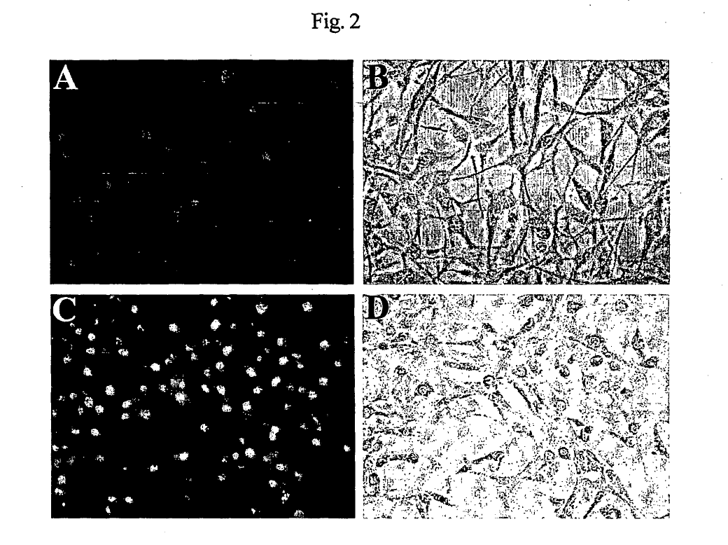

FIG. 2 is a series of micrographs showing the staining and morphology of differentiated BMSCs derived from human using the antibody specific for MEF-2, following a co-culture with growth factors in the absence (A) or presence (C) of IGF-1. In FIG. 2, panels A and B are the results from the culture in the presence of bFGF and BMP2, and panels C and D, in the presence of bFGF, BMP2 and IGF-1. Further, panels A and C are the results from the immunofluorescence staining by MEF2, and panels B and D show corresponding phase contrast images thereof. All images were observed under 40× objective. As shown in FIG. 2, the maximum quantity of cardiomyogenic cells is obtained in the presence of 2 ng/ml of IGF-1. Further, the expression of MEF2 in the cells co-cultured with IGF-1 is stronger than that cultured without IGF-1, suggesting enhanced cardiomyogenic cellular characteristics. According to the present invention, the highest yield of cardiomyogenic cells which have the characteristics of cardiomyocyte cell lineage can be obtained by this method.

In view of the foregoing results, the rate and amount that BMSCs become cardiomyogenic cells in culture can be regulated and maximized by modulating the environment in which the cells are cultured. According to the transplantation method of the invention, it is preferable that at least 50% of the transplanted cells be cardiomyogenic cells. A higher percentage cardiomyogenic cells will result in increased incorporation of implanted cells. Thus, it is preferable that at least about 50%, 75%, 85%, 90% or 95% or more of the cells be cardiomyogenic cells.

In the method of the present invention, suitable factors or conditions are those that specifically induce one cell type (e.g., cardiomyocytes).

### EXAMPLE 2

### BMSCs from Humans and other Mammals

Human BMSCs are also known in the art to be capable of producing cardiomyocyte (Pittenger et al., Science 284: 143-147, 1999). BMSCs from other mammals (e.g., humanized pig BMSCs) can also be used in the methods of the invention (Levy et al., Transplantation 69: 272-280, 2000).

### INDUSTRIAL APPLICABILITY

As apparent from the above description, when BMSCs are cultured in the media containing IGF-1 under the condition to induce the cells toward cardiomyogenic cells, the highest yield of the cells to be transplanted into mammal cardial tissue which have the characteristics of cardiomyocyte cell lineage mostly can be obtained. Further, the cells produced as such can be used for treating a disorder characterized by insufficient cardiac function.

1. A method for producing cells for transplantation into myocardial tissue of a mammal comprising the steps:
(a) providing bone marrow stem cells that have not been immortalized;
(b) culturing said bone marrow stem cells in a culture medium containing IGF-1 (Insulin-like Growth Factor-1) under conditions that induce said cells to differentiate into cardiomyogenic cells;
(c) monitoring the differentiation state of the cells of step (b); and
(d) collecting the cells of step (b) when at least about 50% of said cells are cardiomyogenic cells.

2. The method of claim 1, wherein said bone marrow stem cells are derived from the mammal to be treated.

3. The method of claim 1, wherein said mammal is a human.

4. The method of claim 1, wherein said step (d) is performed when at least 50% and as many as 80% of said cells of step (b) are cardiomyogenic cells.

5. The method of claim 1, wherein the concentration of IGF-1 ranges from 0.1 to 25 ng/ml.

6. Cells for transplantation into myocardial tissue of a mammal, which are produce by culturing bone marrow stem cells that have not been immortalized in a culture medium containing IGF-1 (Insulin-like Growth Factor-1) under conditions that induce said cells to differentiated into cardiomyogenic cells and collecting the cells.

7. The cells of claim 6, wherein said bone marrow stem cells are derived from the mammal to be treated.

8. The cells of claim 6, wherein said mammal is a human.

9. The cells of claim 6, wherein the concentration of IGF-1 ranges from 0.1 to 25 ng/ml.

10. A pharmaceutical composition for transplantation into myocardial tissue of a mammal diagnosed as having a disorder characterized by insufficient cardiac function to treat the mammal, which comprises:
(a) cardiomyocytes or cardiomyocyte progenitors produced according to claim 1;
(b) endothelial cells or endothelial cell progenitors; and
(c) vascular smooth muscle cells or vascular smooth muscle cell progenitors.

11. The composition of claim 10, wherein the ratio of cardiomyocyte progenitors:endothelial cell progenitors:vascular smooth muscle cell progenitors is 10:1:1.

12. The composition of claim 10, wherein said mammal is a human.
